(19) United States
(12) Patent Application Publication
(10) Pub. No.: US 2019/0209759 A1
(43) Pub. Date: Jul. 11, 2019

(54) VENTRICULAR ASSIST DEVICES

(71) Applicants: Children`s Medical Center Corporation, Boston, MA (US); President and Fellows of Harvard College, Cambridge, MA (US)

(72) Inventors: Nikolay V. Vasilyev, Newton, MA (US); Christopher J. Payne, Cambridge, MA (US)

(21) Appl. No.: 16/312,561

(22) PCT Filed: Jun. 23, 2017
(86) PCT No.: PCT/US17/39106

§ 371 Date: Dec. 21, 2018

Related U.S. Application Data
(60) Provisional application No. 62/354,196, filed on Jun. 24, 2016.

(51) Int. Cl. A61M 1/12; A61M 1/10

(52) U.S. Cl. CPC A61M 1/122; A61M 1/1086; A61M 1/1068; A61M 1/1062

(57) ABSTRACT

A ventricular assist device includes a rigid, elongated shaft; an anchor assembly attached to a first end of the elongated shaft; a brace attached to a second end of the elongated shaft, the brace having a surface facing the anchor assembly; and one or more actuators attached to the brace and disposed adjacent the first surface of the brace.

### CLAIM OF PRIORITY

This application claims priority to U.S. Patent Application Ser. No. 62/354,196, filed on Jun. 24, 2016, the entire contents of which are hereby incorporated by reference.

### STATEMENT AS TO FEDERALLY SPONSORED RESEARCH

This invention was made with Government support under Grant No. PR141716 Discovery Award awarded by the Department of Defense Congressionally Directed Medical Research Programs (CDMRP). The Government has certain rights in the invention.

### FIELD OF THE INVENTION

The invention relates to ventricular assist devices and methods.

### BACKGROUND OF THE INVENTION

End-stage heart failure can be treated by heart, lung, or heart-lung transplantation. When no suitable donor organ is available, a variety of approaches can be used for temporary or long-term therapeutic treatments. For instance, treatment can include augmenting heart function through mechanical circulatory support, e.g., using ventricular assist devices such as pulsatile pumps that mimic the pumping function of the heart or continuous flow pumps. Other therapeutic solutions to heart failure can include medical therapy, surgical reconstruction of the ventricle, passive ventricular constraint, cardiac resynchronization therapy, or cellular or dynamic cardiomyoplasty.

### SUMMARY OF THE INVENTION

The disclosure is based, at least in part, on the discovery that ventricular assist devices can augment the function of the diseased ventricle, such as the right ventricle or the left ventricle, by applying a compressive force to the ventricle free wall from the external surface of the ventricle free wall. The ventricular assist devices are rigidly braced to the septum, and include actuators disposed external to the ventricle free wall. Actuation of the actuators causes a compressive force to be applied to the ventricle free wall at multiple points or in a continuous zone, which results in the ventricle free wall moving towards the septum. This motion of the ventricle free wall enhances the function of the targeted diseased ventricle and increases the blood flow through the ventricle, while preserving function of the other ventricle.

In an aspect, a ventricular assist device includes a rigid, elongated shaft; an anchor assembly attached to a first end of the elongated shaft; a brace attached to a second end of the elongated shaft, the brace having a surface facing the anchor assembly; and one or more actuators attached to the brace and disposed adjacent the first surface of the brace.

Embodiments can include one or more of the following features.

The one or more actuators are configured to expand towards the anchor assembly when actuated.

The brace has an arc shape.

The anchor assembly comprises an anchor having multiple, collapsible arms. The collapsible arms have a first configuration in which the collapsible arms are collapsed along a central post of the anchor and a second configuration in which the collapsible arms are disposed away from the central post of the anchor. The anchor comprises a central post. The anchor assembly comprises a disc attached to the central post of the anchor.

The one or more actuators comprise inflatable actuators.

The one or more actuators comprise actuators configured to expand in one or more dimensions when actuated.

The one or more actuators comprise actuators configured to bend in one or more dimensions when actuated.

The one or more actuators comprise pneumatic artificial muscle.

The ventricular assist device includes a ring disposed along the shaft, wherein the shaft passes through a central opening of the ring. The ventricular assist device includes a sealing component. A first side of the sealing component is attached to the ring and a second side of the sealing component is attached to the shaft. The ventricular assist device includes a recoil component connected to the brace and to the ring, wherein the recoil component is configured to apply a recoil force to the ring. The recoil force is in a direction opposite to a direction of the expansion of the one or more actuators. The recoil component comprises one or more of a spring and an elastic band.

The ventricular assist device includes a control system configured to control actuation of the one or more actuators. The control system is configured to control actuation of the one or more actuators based on a signal indicative of heart function. The signal indicative of heart function comprises one or more of a pacemaker signal, an electrocardiography signal, a pressure in a ventricle of the heart, a pressure in an atrium of the heart, and a pressure in a great vessel.

The brace includes multiple sub-pieces connected by a narrow, flexible member, e.g., a metal wire or string, e.g., of nitinol or stainless steel.

In an aspect, a ventricular assist device inserted in a heart of a patient includes an anchor assembly secured to a septum of the heart. The ventricular assist device includes an elongated shaft. A first end of the elongated shaft is attached to the anchor assembly, a length of the elongated shaft is disposed in a ventricle of the heart, and a second end of the elongated shaft is disposed outside of a free wall of the ventricle. The ventricular assist device includes a brace attached to the second end of the elongated shaft; and one or more actuators attached to the brace and disposed between the brace and the free wall of the ventricle.

Embodiments can include one or more of the following features.

The one or more actuators are configured to apply a compressive force to the free wall of the ventricle when actuated. The compressive force applied to the free wall of the ventricle is sufficient to cause the free wall of the ventricle to move toward the septum. The compressive force applied to the free wall of the ventricle is sufficient to cause the ventricle to shorten along an axis of the ventricle.

The ventricle is a first ventricle. The anchor assembly includes an anchor disposed along a side of the septum facing a second ventricle of the heart; a disc disposed along a side of the septum facing the first ventricle of the heart; and a central post connecting the anchor and the disc.

The elongated shaft passes through an incision in the free wall of the ventricle. The ventricular assist device includes a ring disposed in the incision, wherein the shaft passes through a central opening of the ring. The ventricular assist device includes a recoil component connected to the brace and to the ring, wherein the recoil component is configured to apply a recoil force to the ring.

The one or more actuators comprise inflatable actuators.

The ventricle comprises the right or left ventricle.

The brace includes multiple sub-pieces connected by a narrow, flexible member, e.g., a wire of metal, e.g., nitinol or stainless steel, or a string.

In an aspect, a method of using a ventricular assist device disposed in a heart of a patient includes actuating one or more actuators disposed outside of a free wall of a ventricle of the heart to apply a compressive force to the free wall of the ventricle. The one or more actuators are attached to a brace that is coupled to a ventricular septum of the heart. Application of the compressive force causes one or more of (i) the free wall of the ventricle to move towards the septum and (ii) the ventricle to shorten along an axis of the ventricle. The method includes de-actuating the one or more actuators to remove the compressive force from the free wall of the ventricle.

Embodiments can include one or more of the following features.

Actuating the one or more actuators comprises inflating the one or more actuators.

Actuating the one or more actuators comprises expanding the one or more actuators in one or more dimensions.

Actuating the one or more actuators comprises bending the one or more actuators in one or more dimensions.

The one or more actuators comprise pneumatic artificial muscle and wherein actuating the one or more actuators comprises contracting the pneumatic artificial muscle.

The brace is rigidly coupled to the septum.

The method includes controlling the actuating and de-actuating of the one or more actuators based on a signal indicative of heart function. The method includes actuating the one or more actuators during diastole. The method includes de-actuating the one or more actuators during systole. The signal indicative of heart function comprises one or more of a pacemaker signal, an electrocardiography signal, and a pressure in a ventricle of the heart.

In an aspect, a method of inserting a ventricular assist device into a heart of a patient includes securing an anchor assembly to a septum of the heart; and attaching a first end of an elongated shaft to the anchor assembly. A brace is attached to a second end of the elongated shaft. The brace remains outside of a free wall of the ventricle. One or more actuators attached to the brace are disposed between the brace and the free wall of the ventricle.

Embodiments can include one or more of the following features.

Inserting the elongated shaft into the ventricle of the heart comprises inserting the elongated shaft through a ring inserted into an incision in the free wall of the ventricle.

The ventricular assist devices and methods described herein can have one or more of the following advantages. The ventricular assist devices apply a compression force to a diseased right or left ventricle free wall from outside of the heart, which enables the function of the diseased ventricle to be augmented without having any significant effect on the function of the other ventricle and other chambers of the heart. The compression force is applied to the free ventricle wall at multiple points or in a continuous zone, thus reducing the occurrence of outward bulging in portions of the ventricle free wall to which the force is not directly applied. Insertion of the ventricle assist devices is a rapid procedure that can be performed in a beating heart, thus making these devices well suited for use in emergency situations and allowing these devices to be implanted without open-heart surgery or cardiopulmonary bypass procedures.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIGS. 1A and 1B are diagrams of a ventricular assist device in diastole.

FIGS. 1C and 1D are diagrams of a ventricular assist device in systole.

FIG. 2 is a diagram of a ventricular assist device.

FIG. 3 is a photograph of a ventricular assist device.

FIGS. 4A and 4B are examples of anchors.

FIGS. 5A and 5B are diagrams of ventricular assist devices for the right ventricle and left ventricle, respectively.

FIGS. 6A and 6B are photographs of a brace with de-actuated and actuated actuators, respectively.

FIG. 7 is a diagram of a ventricular assist device with recoil components.

FIG. 8 is a photograph of a ventricular assist device with recoil components.

FIG. 9 is a diagram of a ring.

FIGS. 10A-10M are diagrams of a process of inserting a ventricular assist device into the heart of a patient.

FIG. 11 is a flow chart of a process of inserting a ventricular assist device into the heart of a patient.

FIG. 12 is a block diagram of a control system.

FIG. 13 is a diagram of a ventricular assist device with a McKibben actuator.

FIG. 14 is a diagram of a ventricular assist device with extendible actuators.

FIGS. 15A and 15B are diagrams of a foldable ventricular assist device.

FIG. 16A is a plot of force profiles for different systolic timings as a percentage of the total cardiac cycle for a right ventricular assist device.

FIG. 16B is a plot of peak reaction forces observed for different systolic timings for a right ventricular assist device.

FIG. 16C is a plot characterizing the pulmonary flow output for the actuation of the ventricular assist device at different systolic timing periods and delay periods for a right ventricular assist device.

FIG. 16D is a plot showing the contribution of the actuator pairings on pulmonary flow rate for a right ventricular assist device.

FIGS. 17A-17C are plots showing the pulmonary flow rate, aortic flow rate, and end tidal CO2, respectively, versus time at baseline, heart failure, and with an actuated right ventricular assist device.

FIGS. 17D-17G are plots showing the pulmonary flow rate, aortic flow rate, peak right ventricle pressure, and end diastolic right ventricle pressure at baseline, heart failure, and with an actuated right ventricular assist device.

FIGS. 18A-18C are plots showing the aortic flow rate, left ventricle pressure, and left atrial pressure versus time at baseline, heart failure, and with an actuated left ventricular assist device.

FIGS. 18D-18G are plots showing aortic flow rate, pulmonary flow rate, peak left ventricle pressure, and end diastolic left ventricle pressure at baseline, heart failure, and with an actuated left ventricular assist device.

FIG. 19 is a plot of the simulated performance of a foldable ventricular assist device versus a one-piece ventricular assist device.

FIG. 20 is a plot of the performance of a foldable ventricular assist device over time.

FIG. 21 is a plot of the performance of a foldable ventricular assist device versus a one-piece ventricular assist device.

### DETAILED DESCRIPTION

Ventricular assist devices augment the blood flow in a ventricle of the heart by approximating the ventricle free wall to the septum. The ventricular assist devices are rigidly braced to the septum, and include actuators disposed external to the ventricle free wall. Actuation of the actuators causes a compressive force to be applied to the ventricle free wall at multiple points or in a continuous zone, which results in the ventricle free wall moving towards the septum. This motion of the ventricle free wall enhances the function of the ventricle and increases the blood flow through the right ventricle. Because the ventricular assist devices are rigidly braced to the septum, the function of the ventricle can be enhanced with no significant impact on the function of any other chamber of the heart.

### Structure of Ventricular Assist Devices

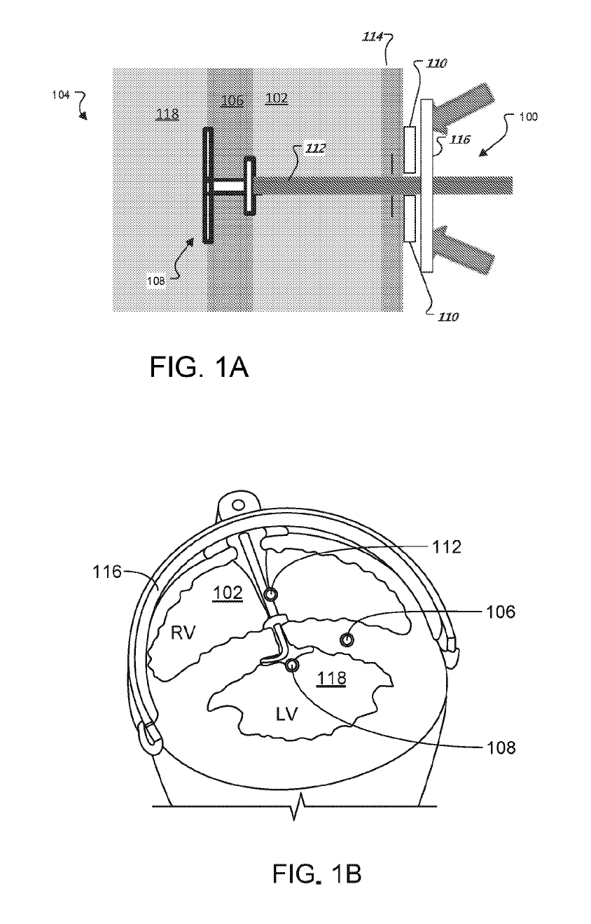

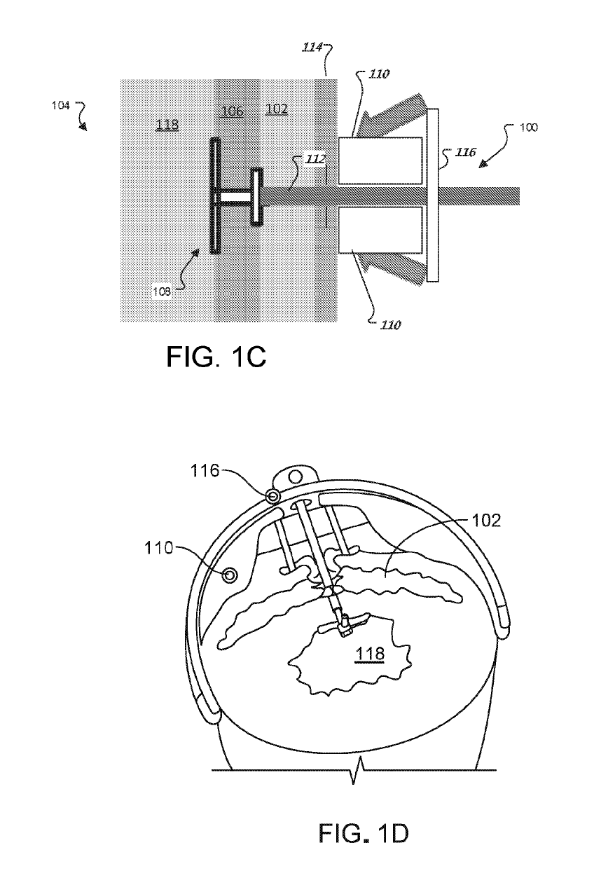

Referring to FIGS. 1A-1D, an example of a ventricular assist device 100 augments blood flow in the right ventricle 102 of a patient's heart 104. Although the example shown in FIGS. 1A-1D is for use in the right ventricle, the ventricular assist devices of this disclosure can also be used in the left ventricle. FIGS. 1A and 1B show the ventricular assist device 100 in diastole; FIGS. 1C and 1D show the ventricular assist device 100 in systole.

The ventricular assist device 100 includes one or more soft actuators 110 positioned external to the right ventricle free wall 114. The actuators 110 can be actuated to apply a compressive mechanical force to the right ventricle free wall 114. In some examples, the compressive mechanical force causes the right ventricle free wall 114 to be approximated to the ventricular septum 106, thus augmenting the squeezing of blood out of the right ventricle 102. In some examples, the compressive mechanical force causes the right ventricle 102 to be shortened along its axis, thus augmenting the squeezing of blood out of the right ventricle. For instance, the actuators 110 can be actuated during diastole to augment the pumping action of the right ventricle 102. During systole, the actuators 110 are de-actuated, removing the force from the right ventricle free wall 114 and transmitting the reaction forces to the ventricular septum 106, allowing the right ventricle 102 to fill with blood.

To cause motion of the ventricle free wall 114 relative to the ventricular septum 106 (which sometimes also refer to as the septum 106), the ventricular assist device 100 is anchored to the septum 106 by an anchor assembly 108. A rigid shaft 112 connects the anchor assembly 108 to an external brace 116, which bears the actuators 110. Because the position of the septum 108 relative to the external brace 116 is fixed, the actuation of the actuators 110 causes the ventricle free wall 114 to be approximated toward the septum 106, thus augmenting blood flow through the ventricle 102. In addition, the rigid bracing of the ventricular assist device 100 against the septum 106 enables the ventricle function to be augmented without significant effect on the function of the other ventricle 118. For instance, in the specific example of FIGS. 1A and 1B, the ventricular assist device 100 augments the function of the right ventricle 102 without significant effect on the function of the left ventricle 118.

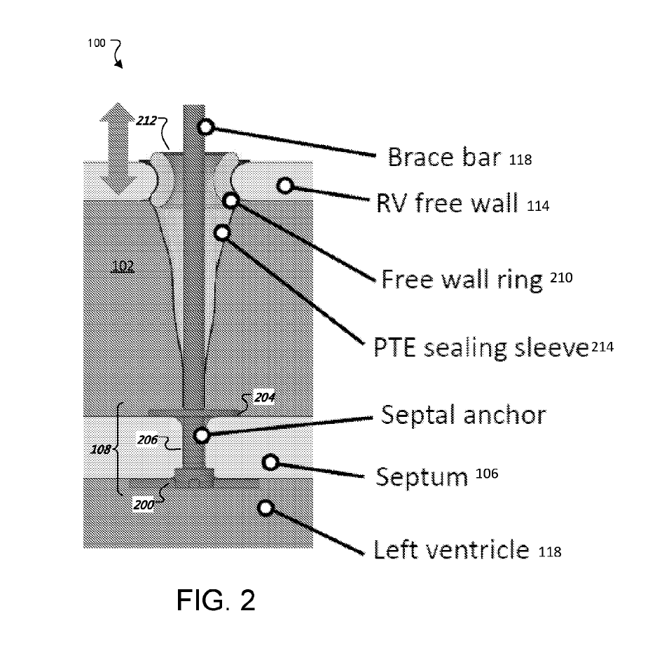

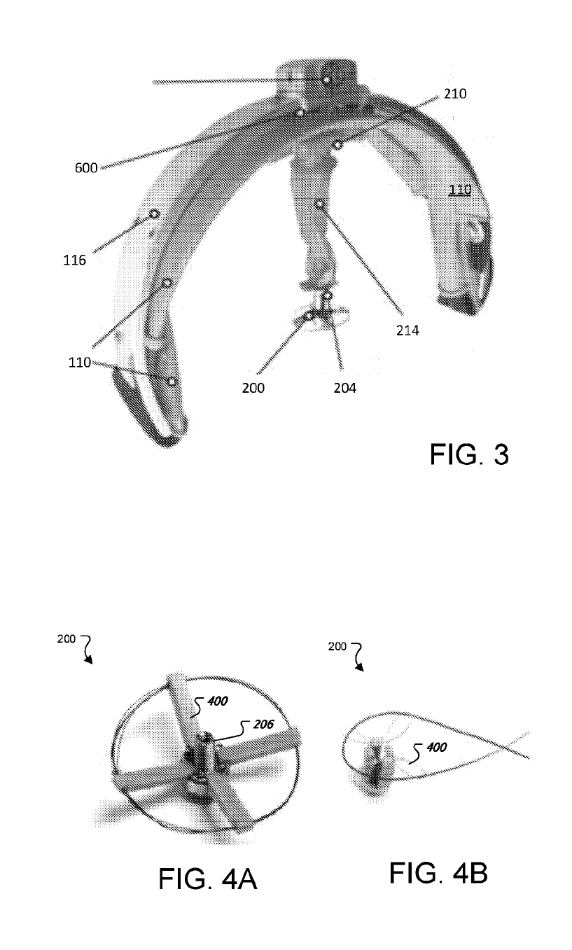

Referring to FIGS. 2 and 3, the anchor assembly 108 that anchors the ventricular assist device 100 to the septum 106 includes an anchor 200 positioned on the surface of the septum 106 facing the left ventricle 118. A central post 206 of the anchor penetrates through the septum 106 and into the right ventricle. A disc 204 is attached to a central post 206 of the anchor 200 and positioned on the surface of the septum 106 facing the right ventricle 102. For instance, the disc can be screwed onto the central post 206, attached via a press-fit connection, or attached in another way. The anchor assembly 108 thus sandwiches the septum 106 between the anchor 200 and the disc 204, bracing the ventricular assist device 100 to the septum.

In some examples, the anchor 200 can be expandable after insertion into the heart. For instance, referring to FIGS. 4A and 4B, the anchor 200 can have multiple arms 400 that extend radially from the central post 206 of the anchor. During insertion of the anchor 200 into the heart, the arms 400 are folded against the central post 206 of the anchor to reduce the size of the incision needed for insertion. After insertion, the arms 400 can be unfolded, e.g., like an umbrella, causing the anchor 200 to flatten against the other ventricle surface of the septum 106 (e.g., the left ventricle surface, for a ventricular assist device that augments blood flow in the right ventricle). Further description of the insertion and deployment of the anchor 200 is provided below. In some examples, the arms 400 of the anchor can be solid pieces of material (FIG. 4A). In some examples, the arms 400 of the anchor can be metal wires, e.g., nitinol wires, with each wire being formed into a round or oval shape (FIG. 4B). In some examples, the arms 400 of the anchor can be polyether-ether-ketone (PEEK) hinge elements coupled with a nitinol spring. In some examples, a thin membrane made of, e.g., Dacron®, elastic polymer, or another material, can be inserted into the umbrella-like structure formed by the arms 400 of the anchor, in order to help prevent shunt blood flow between the ventricles (e.g., from the left ventricle to the right ventricle) along the central post 206 of the anchor 200.

Additional description of anchors can be found in U.S. application Ser. No. 14/377,560, the contents of which are incorporated here by reference in their entirety.

Referring again to FIGS. 2 and 3, the shaft 112 connecting the anchor assembly 108 to the external brace 116 can be formed of a rigid material, such as a metal (e.g., stainless steel, titanium, or another biocompatible metal), a rigid biocompatible polymer (e.g., polyethylene polyaryletherketone, polyether ether ketone, delrin, hytrel, crastin, zytel, or another biocompatible polymer), or another rigid material. The shaft 112 can have a diameter of between about 1 mm and about 4 mm, e.g., about 1 mm, about 2 mm, about 3 mm, about 4 mm, or another diameter.

The external end of the shaft 112 connects to the brace 116, which is rests on or near the exterior surface of the ventricle free wall 114. The brace 208 can have a low profile, e.g., a thickness of between about 1 mm and about 3 mm, e.g., between about 1.5 mm and about 2.5 mm, e.g., about 1.5 mm, about 2 mm, about 2.5 mm, or another thickness. The brace can be formed of a rigid material, such as a metal (e.g., stainless steel, titanium, or another biocompatible metal), a rigid biocompatible polymer (e.g., polyethylene, polyaryletherketone, polyether ether ketone, delrin, hytrel, crastin, zytel, or another biocompatible polymer), or another rigid material.

The brace 116 houses the one or more actuators 110. For instance, the brace 116 can house one, two, four, eight, or another number of actuators 110. The actuators 110 are mechanical actuators that expand or elongate in one or more dimensions when actuated, e.g., during systole, thus causing the ventricle free wall 114 to be approximated towards the septum 106. When the actuators 110 are de-actuated, e.g., during diastole, the actuators 110 have a low profile and lie close to the inner surface of the brace 116, allowing the brace to fit closely to the exterior surface of the ventricle free wall 114.

The brace can be shaped specifically for use with the right ventricle or the left ventricle, e.g., to account for differing anatomy and mechanics of each ventricle. The left ventricle wall is thick (e.g., about 20-30 mm in thickness) and has an approximately conical profile. The right ventricle wall is thinner (e.g., about 5-8 mm in thickness) and is wrapped around the left ventricle.

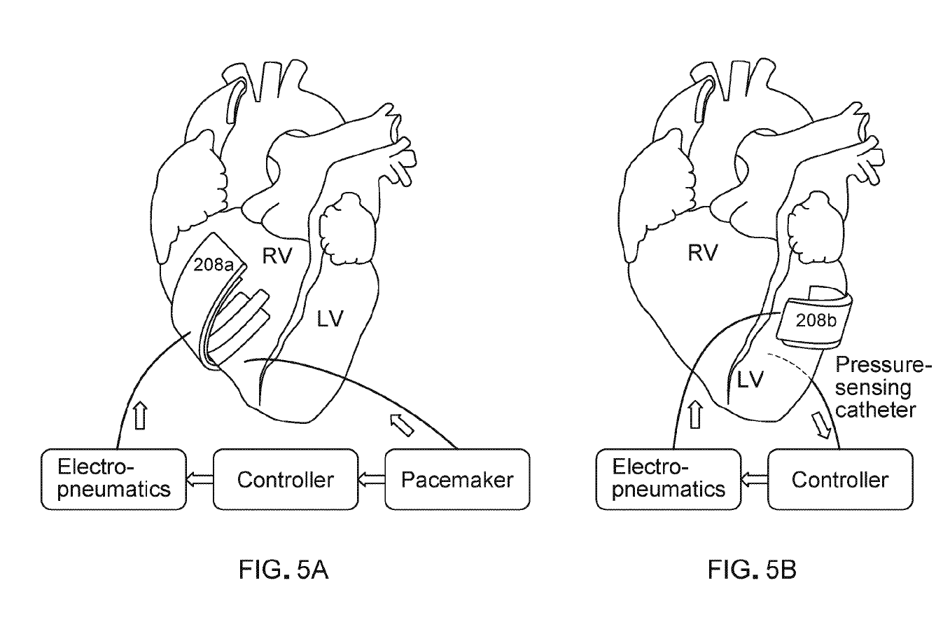

Referring also to FIG. 5A, a brace 208a is designed for use with the right ventricle, can have a curved shape, such as an arc of between about 190° and about 220°, such as 190°, 200°, 205°, 210°, 220°, or another arc. For instance, the right ventricle brace 208a can have a curvature that substantially matches the curvature of the exterior surface of the right ventricle free wall 114. In use, the right ventricle brace 208a can be oriented along the length of the right ventricle spanning from the heart apex to the distal region of the outflow tract. Referring also to FIG. 3, multiple soft actuators 110, such as four actuators, are housed within the right ventricle brace 208a. Two actuators are mounted centrally, adjacent to the shaft 112 of the brace, and the other two actuators are mounted distally at either side of the shaft 112. The four actuators 110 housed in the right ventricle brace 208a provide compression of a large region of the right ventricle free wall in systole to increase or maximize systolic ejection fraction. The right ventricle is a relatively compliant structure, and when a diseased right ventricle is externally compressed, localized distention can occur to accommodate the increased blood pressure. This distention reduces the amount of blood that is ejected through the pulmonary artery and reduces the efficacy of external loading. The use of multiple actuators can constrain the right ventricle in systole, thus increasing blood ejection.

Referring to FIGS. 3 and 5B, a brace 208b designed for use with the left ventricle has a curved shape with a smaller art, such as an arc of between about 140° and about 160°, such as 140°, 150°, 160°, or another arc. In use, the left ventricle brace 208b can be oriented to be parallel with the heart base to accommodate the conical profile of the left ventricle. In this orientation, only two centrally mounted actuators 110 are used to provide effective compression of the left ventricle free wall towards the septum. Since the left ventricle free wall is thicker and relatively stiff as compared to the right ventricle free wall, the left ventricle free wall is less prone to localized distention when externally loaded at a localized area and thus the additional distally located actuators are not used.

In some examples (as shown in FIG. 3), the brace 116 can house multiple balloons actuators, such as rubber or latex balloons, that inflate when actuated. In some examples, the brace 116 can house a pneumatic actuator, such as a McKibben actuator or an actuator based on a McKibben actuator. The actuator can include an elastic bladder and a mesh braid to provide contractile actuation when inflated. In some examples, the brace 116 can house extendible actuators that extend in one dimension when actuated.

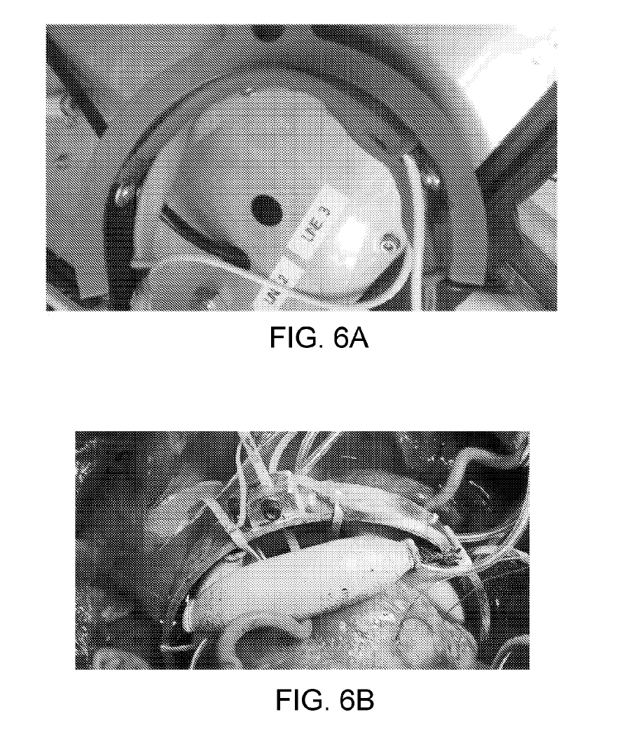

Referring to FIGS. 6A and 6B, a side view of the brace 116 shows the operation of four balloon actuators 110. Referring specifically to FIG. 6A, in a deflated state, e.g., during diastole, the actuators 110 have a low profile and substantially conform to the arc shape of the inner surface of the brace 116. Referring to FIG. 6B, when actuated, e.g., during systole, the actuators 110 inflate, increasing in volume. When inserted in the heart of a patient, the brace 116 is rigidly fixed to the septum 106 by the anchor assembly and thus cannot move when the actuators 110 inflate. As a result, the inflation of the actuators 110 causes a force to be applied to the ventricle free wall 114, approximating the ventricle free wall 114 toward the septum 106. This motion of the ventricle free wall 114 causes the volume in the ventricle 102 to decrease and augments blood flow from the ventricle 102.

The use of actuators 110 that apply a force to the ventricle free wall 114 at more than a single point can provide effective augmentation of blood flow from the ventricle 102. For instance, by applying a force at multiple points or in a continuous zone, the ventricle free wall 114 is approximated to the septum 106 at more than just a single point, and thus the ventricle free wall 114 is prevented from ballooning out at other points.

Referring again to FIG. 2, the shaft 112 passes through an incision in the ventricle free wall 114. To enable the ventricle free wall 114 to move freely relative to the shaft 112 as the actuators 110 are actuated and deactuated, a ring 210 is placed in the incision in the ventricle free wall 114. The ring 210 can be held in place by a suture, such as a purse string suture. The shaft 112 passes through a central opening 212 in the ring 210. The diameter of the central opening 212 is greater than the diameter of the shaft 112, thus allowing the ring 210 (and hence the ventricle free wall 114) to slide smoothly relative to the shaft 112.

To prevent blood from leaking out of the ventricle 102 and external contaminants (e.g., blood clots) from entering the right ventricle through the central opening 212 in the ring 210, the ring 210 is sealed by a sealing sleeve 214. The sealing sleeve is sealed to the ring 210 and onto the shaft 112, e.g., to the central region of the shaft 112 or at the septum end of the shaft 112, so that there is no fluid pathway between the interior of the right ventricle 102 and the exterior of the heart. The sealing sleeve 214 can be a flexible, biocompatible polymer, such as polyethylene terephthalate (PTE), nylon, or another flexible, biocompatible polymer.

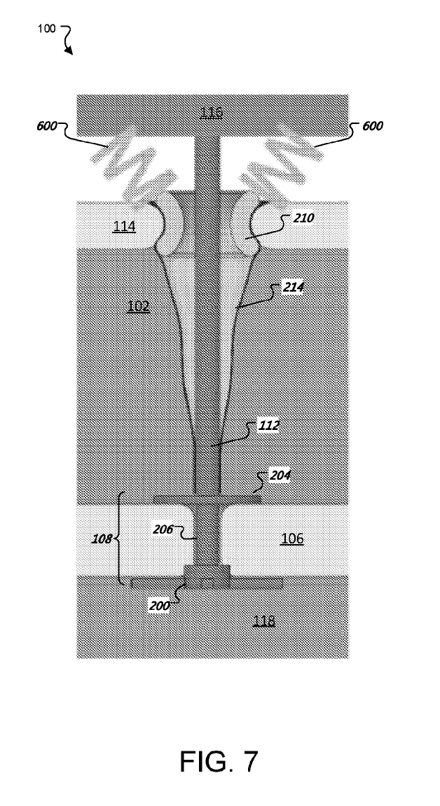

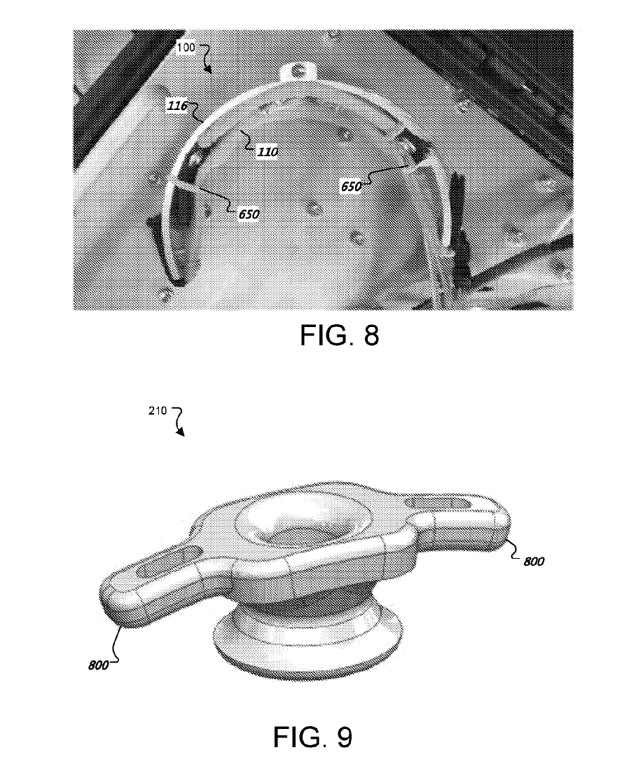

Referring to FIGS. 3, 7 and 8, in some examples, the ventricular assist device 100 can include one or more recoil components 600 that are affixed to both the brace 116 and the ring 210. For instance, referring also to FIG. 9, the ring 210 can include lateral extensions 800 each having an attachment point 802, such as a hole, where a recoil component 600 can be attached. In some examples, the ventricular assist device 100 can include one or more recoil components 650 that are wrapped around the actuators.

The recoil components 600, 650, e.g., elastic bands or springs, apply a recoil force to the ring 210 and to the actuators 110, respectively. The recoil force applied by the recoil components 600 is substantially opposite in direction to the force applied to the right ventricle free wall 114 by actuation of the actuators 110.

Without recoil components 600, the actuators 110 can in some cases be slow to deflate during systole, thus hindering the refilling of the right ventricle 102. The recoil force applied by the recoil components 600 to the ring 210 during systole pulls the ring 210, and hence the right ventricle free wall 114, back towards the brace 116, enabling more complete refilling of the right ventricle 102. Similarly, the recoil force applied by the recoil components 650 to the actuators 110 pulls the actuators 110 back towards the brace 116. During diastole, the actuators 110 can overcome the recoil force applied by the recoil components 600 in order to apply a compressive force on the right ventricle free wall. For instance, the elastic modulus of the recoil components 600 can be selected such that the actuators 110 exert sufficient force to overcome the recoil force applied by the recoil components 600. In some examples, the actuators 110 exert a compressive force on the right ventricle free wall 114 that is less than about 20 N, such as between about 5 N and about 20 N, e.g., between about 10 N and about 15 N. In some examples, the recoil components 600 apply a recoil force that is between about 50 N/m and about 150 N/m.

In some examples, the actuators 110 are attached directly to the ventricle free wall 114 by sutures, staples, or tissue adhesives. This approach facilitates constant contact between the actuators 110 and the ventricle free wall 114 during systole and diastole and enables the ventricular assist device 100 to provide additional physiological augmentation of the ventricle. During diastole, this approach also enables the device to facilitate recoiling of the ventricle free wall 114 and diastolic filling of the ventricle.

In some examples, actuation of the actuators 110 can cause the actuators 110 to bend in one or more dimensions. For instance, a distal end of each actuator 100 can be bent inwards towards the shaft of the ventricular assist device, thus applying a mechanical force to the ventricle free wall 114 that causes the ventricle to shorten along its long axis.

### Use and Placement of Ventricular Assist Devices

In some examples, the ventricular assist devices described here can be used to provide short term support to patients with severe heart failure, such as post-operative or post-cardiotomy patients. For instance, the ventricular assist devices can be used for a period of less than about 24 hours, e.g., about 3 hours, about 6 hours, about 8 hours, about 12 hours, or another period of time. In some examples, the anchor assembly can remain in the patient's heart on a long term basis, and the other components of the ventricular assist device, such as the shaft and the brace, can be inserted temporarily. In some examples, the ventricular assist devices can remain in the patient's heart on a long term basis for ongoing support.

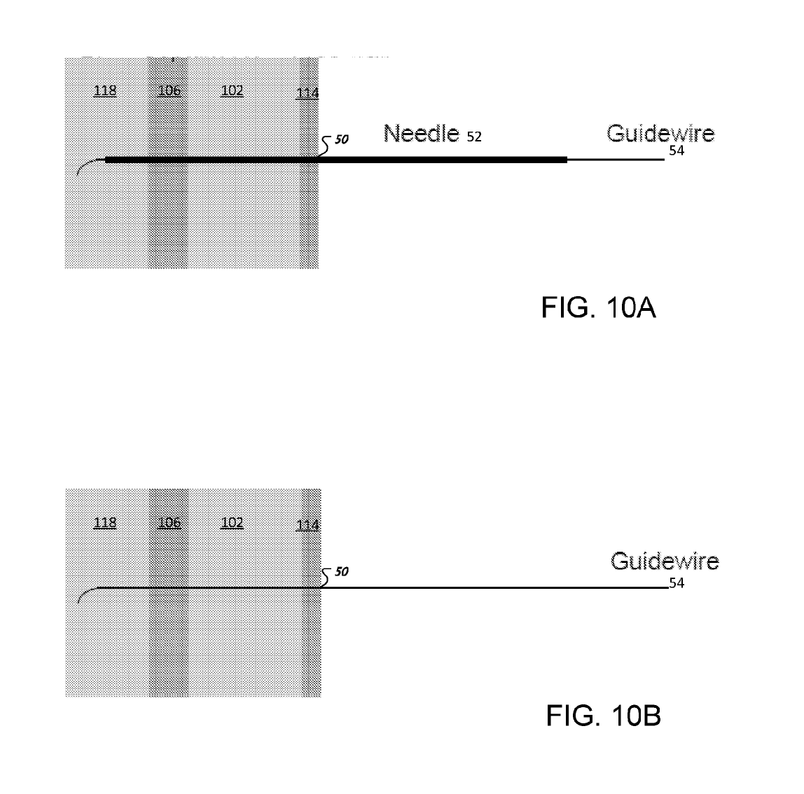

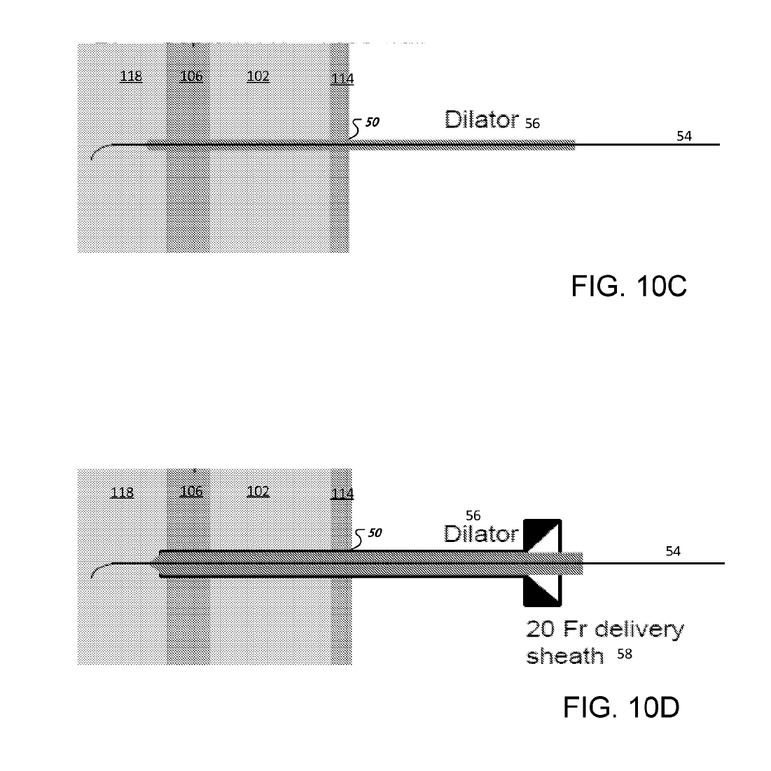

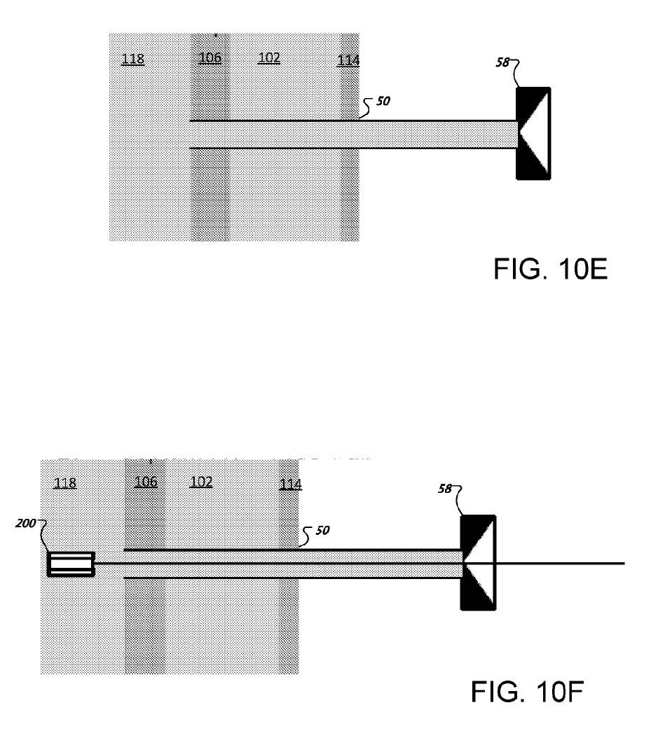

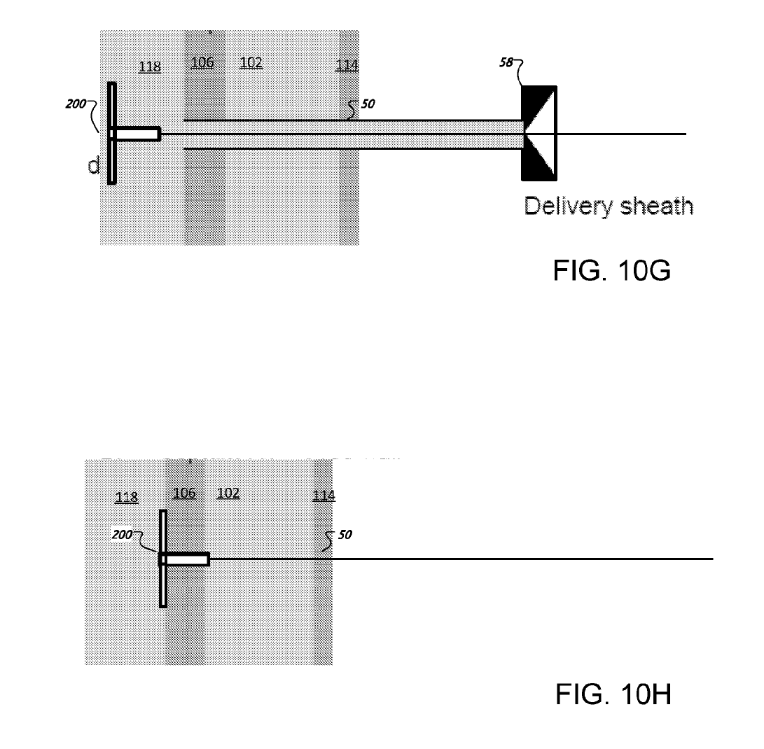

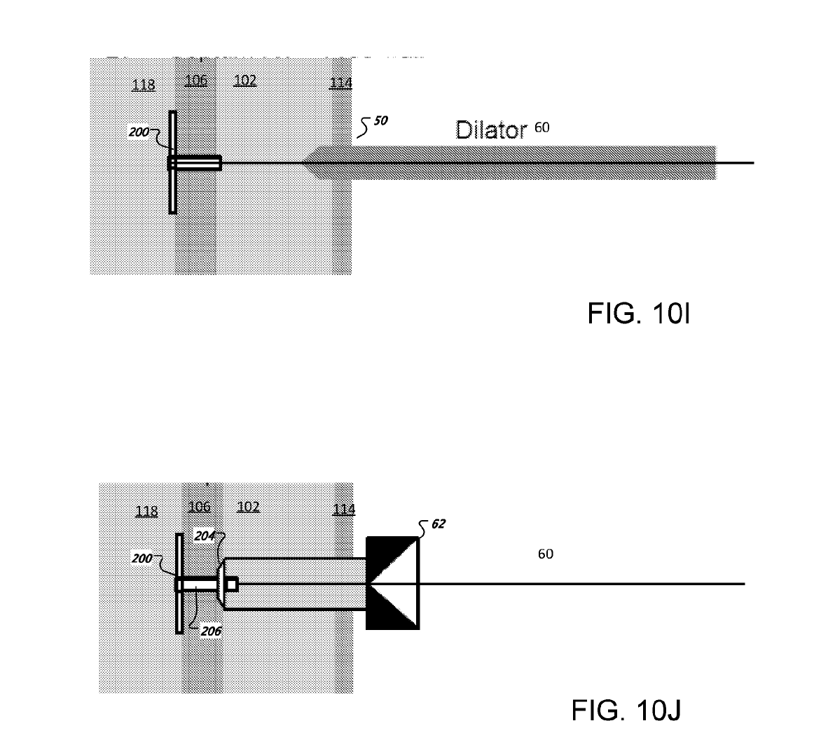

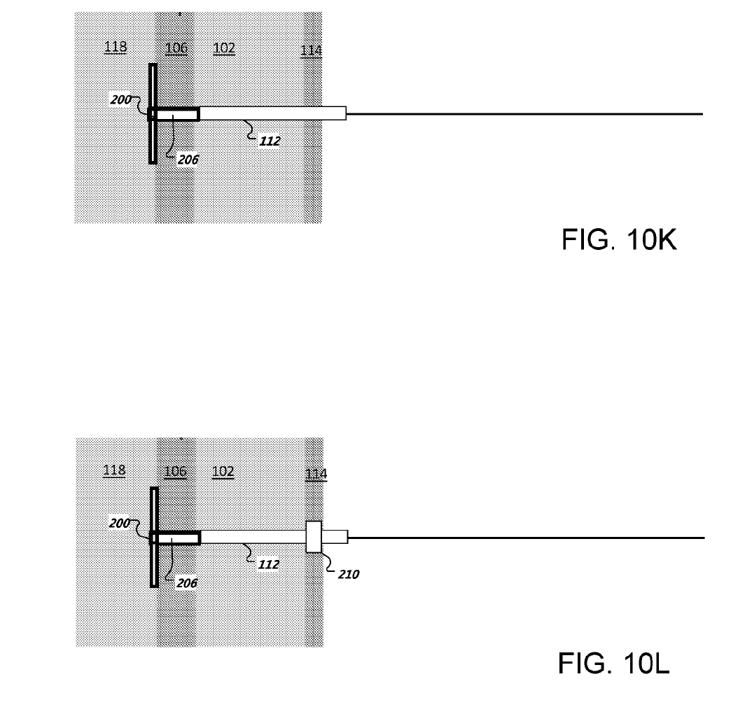

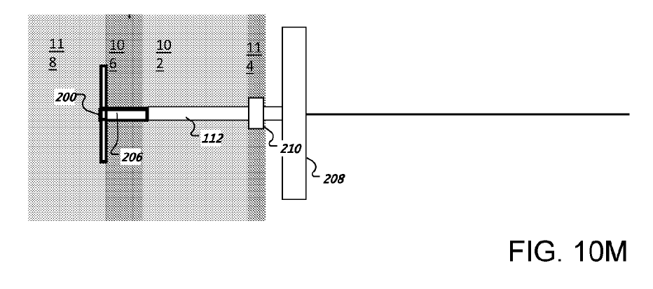

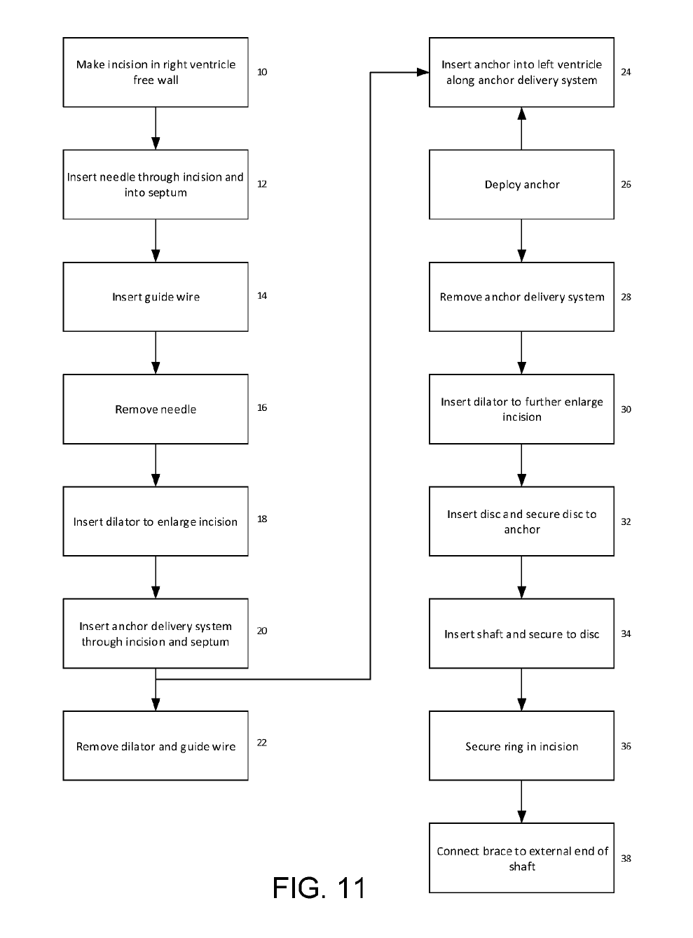

FIGS. 10A-10M and FIG. 11 depict an example process for inserting a ventricular assist device into the heart of a patient. In some examples, the ventricular assist device can be inserted into a beating heart, e.g., under echocardiography guidance. These figures refer to deployment of a right ventricular assist device; access to the heart is reversed for deployment of a left ventricular assist device.

Access to the right ventricle free wall and the ventricular septum into the left ventricle is obtained using the Seldinger technique under echocardiography guidance. Referring to FIGS. 10A and 11, an incision 50 is made in the right ventricle free wall 114 and a purse string suture is formed at the incision (10). A needle 52 is inserted into the incision 50 and through the septum 106 (12). A guide wire 54 is inserted through the needle 52 (14). Referring to FIG. 10B, the needle 52 is removed, leaving only the guide wire 54 in the beating heart (16).

Referring to FIG. 10C, a dilator 56 is inserted along the guide wire 54 and through the septum 106 to create an opening large enough for the anchor to be inserted (18). In some examples, a series of increasingly large dilators can be used. Referring to FIG. 10D, an anchor delivery system 58 is inserted through the incision 50 in the right ventricle free wall 114 and through the septum 106 (20). The anchor delivery system 58 can have a size of between about 18 Fr and about 22 Fr, e.g., 18 Fr, 20 Fr, 22 Fr, or another size. Referring to FIG. 10E, after insertion of the anchor delivery system 58, the dilator 56 and guide wire 54 are removed (22), leaving only the anchor delivery system 58 in the heart.

Referring to FIG. 10F, the anchor 200 is inserted along the anchor delivery system 58 into the left ventricle 202 (24). The anchor 200 can be inserted in a collapsed state, e.g., with the arms of the anchor disposed against the central post of the anchor. Referring to FIG. 10G, the anchor 200 is deployed (26). For instance, the arms 400 of the anchor can be expanded like an umbrella. In some examples, the anchor 200 can be deployed by triggering expansion of the arms 400 using the guide wire 54 or a component of the anchor delivery system 58. In some examples, a locking system can be used to lock the anchor in its deployed configuration, e.g., once the anchor is confirmed to be at the desired position. Referring to FIG. 10H, after anchor insertion and deployment, the anchor delivery system 58 is removed (28), causing the anchor 200 to be pulled against the septum 106, e.g., so that the anchor 200 is substantially flush with the septum 106.

Referring to FIG. 10I, the incision 50 through the right ventricle free wall 114 is further dilated with a second dilator 60 to create an opening large enough for the disc to be inserted (30). In some examples, a series of increasingly large dilators can be used. In some examples, the opening previously created by the dilator 56 is also large enough for the disc to be inserted, and no further dilation is necessary. Referring to FIG. 10J, the disc 204 is inserted into the right ventricle 102 (32) and delivered to the septum 106 by a delivery device 62, such as a trocar. The disc 204 is secured to the central post 206 of the anchor 200, e.g., by screwing the disc 204 onto the central post 206, by way of a press-fit connection, or in another way. In the example shown, the configuration of the disc 204 is not changed after the disc 204 is inserted into the right ventricle 102. In some examples, the disc can be an expandable disc that is deployed into an open configuration after insertion into the right ventricle 102.

Referring to FIG. 10K, the shaft 112 is inserted through the incision 50 in the right ventricle free wall 114 (34) and secured to the disc 204, e.g., by screwing the shaft 112 onto a central post of the disc 204, by way of a press-fit connection, or in another way. The sealing sleeve (not shown) can be connected to the shaft 112 prior to insertion of the shaft 112 into the right ventricle. The shaft 112 extends out of the incision 50 to the exterior of the heart. Referring to FIG. 10L, the ring 210 is threaded along the shaft 112 and secured in place in the incision 50 in the right ventricle free wall (36), e.g., using a purse string suture or another attachment mechanism. The sealing sleeve can be sealed to the ring 210 prior to securing the ring in the incision. In some examples, the ring 210 can first be positioned in the incision 50 and then the shaft 112 can be inserted through the central opening of the ring 210. In some examples, the shaft 112, the ring 210, and the sealing sleeve are a single unit that are positioned in the right ventricle together.

Referring to FIG. 10M, the external end of the shaft 112 is connected to the brace 208 (38a), e.g., by screwing the brace 208 onto the shaft, by way of a press-fit connection, or in another way. In some examples, the shaft 112 that is inserted into the right ventricle 112 is already connected to the brace 208. For instance, the shaft 112 and the brace 208 can be fabricated as a single unit, or can be connected outside the patient's body prior to insertion.

### Actuation Control in Ventricular Assist Devices

A control unit is used to trigger the actuators in the ventricular assist devices described here, typically in synchrony with the patient's heartbeat. In some examples, the control unit is housed on the brace 116. In some examples, the control unit is external to the patient's body and sends signals to the actuators by a wired or wireless connection.

The actuators are generally actuated or triggered based on the natural operation of the patient's heart. For instance, when the patient's heart enters ventricular systole, the actuators are triggered to augment the natural pumping of the right ventricle. When the heart enters ventricular diastole, the actuators are stopped or de-actuated to allow the right ventricle to fill with blood. In some examples, the timing of actuation can be determined based on a signal from a pacemaker. For instance, when the pacemaker signals to the ventricle to contract, the actuators can be actuated. In some examples, the timing of actuation can be determined based on an electrocardiography signal. For instance, when the electrocardiography signal indicates that the heart has entered ventricular systole, the actuators can be actuated. In some examples, the timing of actuation can be determined based on a measured pressure in a ventricle, such as the right ventricle or the left ventricle. For instance, when the right ventricle pressure begins to increase or when the right ventricle pressure increases beyond a threshold value, the actuators can be triggered. In some examples, the timing of actuation can be determined based on a measured pressure in an atrium of the heart, such as the right atrium or the left atrium; or in a great vessel, such as the superior vena cava, the inferior vena cava, a pulmonary artery or vein, or the aorta.

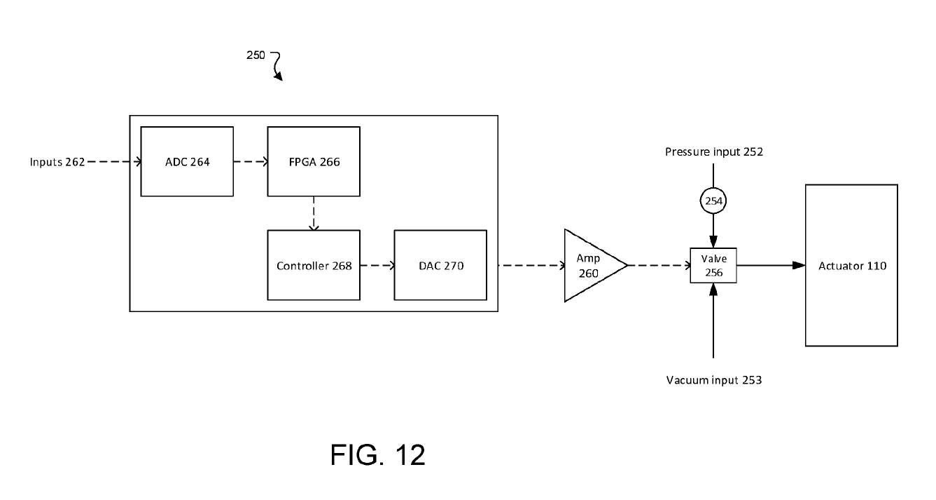

Referring to FIG. 12, a control system 250 triggers the actuators 110. The control system 250 incorporates a pressure input 252 and a vacuum input 253, a pressure regulator 254, one or more state valves 256, computing components 258, power electronics such as a power amplifier 260, and one or more control inputs 262 (e.g., inputs from a pacemaker, ventricle pressure sensor, EKG sensing, or other types of inputs). An input signal (e.g., from a pacemaker, a ventricle pressure sensor, EKG sensing, or another type of input) is acquired through an analog-to-digital converter 264 and read in to an integrated circuit 266, such as a field-programmable gate array (FPGA).

A real-time controller 268 processes the digitized signal from the FPGA and applies a thresholding functioning to trigger the actuator 110, e.g., when the ventricle is in systole. In particular, the controller 268 triggers a digital-to-analog converter 270 to generate a voltage that is passed to the power amplifier 260, which in turn, switches on the valve 256, allowing a pressurizing fluid, such as air or helium, to flow into the actuator 110 or be vacuumed out of the actuator 110. In some examples, a single valve can be arranged so that there are two inputs (fluid (e.g., air or helium) and vacuum) and one output (the actuator) so that the actuator can only receive pressure or vacuum. In some examples, two valves can be used to create a three-state system. In a three-state approach, the actuator 110 can be pressurized, and the pressure can be held in the actuator and then released according to any arbitrary timing. The pressure regulator 254 allows any arbitrary pressure to be set in the actuator 110.

In some examples, the valve 256 is a proportional valve that can control the flow rate of the pressurizing fluid injected into the actuator 110 to allow arbitrary modulation of the contraction rate of the actuator 110. The proportional valve receives a signal from the controller 268 that indicates an aperture for the proportional valve. In some cases, the proportional valve can be programmed to open substantially immediately upon receiving the signal from the controller 268 thus providing a rapid fluid flow rate into the actuator 110. In some cases, the proportional valve can be programmed to open gradually upon receiving the signal from the controller 268 thus accelerating the flow of fluid into the actuator 110. In some cases, the proportional valve can be fully opened upon receiving the signal from the controller 268 and then gradually choked thus decelerating the flow of fluid into the actuator 110. In some examples, the operation of the proportional valve can be tuned such that the contraction of the actuator 110 substantially matches the native contraction of the heart muscle. In some examples, the proportional valve can be used to control the vacuum that is applied to the actuator 110 during diastole.

In some examples, the mechanical response time of the actuator 110 can be tuned by adjusting the properties of the materials of the actuator, such as the materials of the outer sheath of the actuator 110. The outer sheath of the actuator 110 stores elastic energy during systole and allows that stored energy to be released during diastole to enable complete elongation of the actuator 110. The elastic modulus of the outer sheath of the actuator 110 affects the speed with which the actuator can contract during systole and relax during diastole. For instance, forming the outer sheath of the actuator 110 from a higher modulus material, such as a higher modulus elastomer, can slow down the contraction of the actuator 110 during systole and allow for faster relaxation of the actuator 110 during diastole.

### Alternative Structures for Ventricular Assist Devices

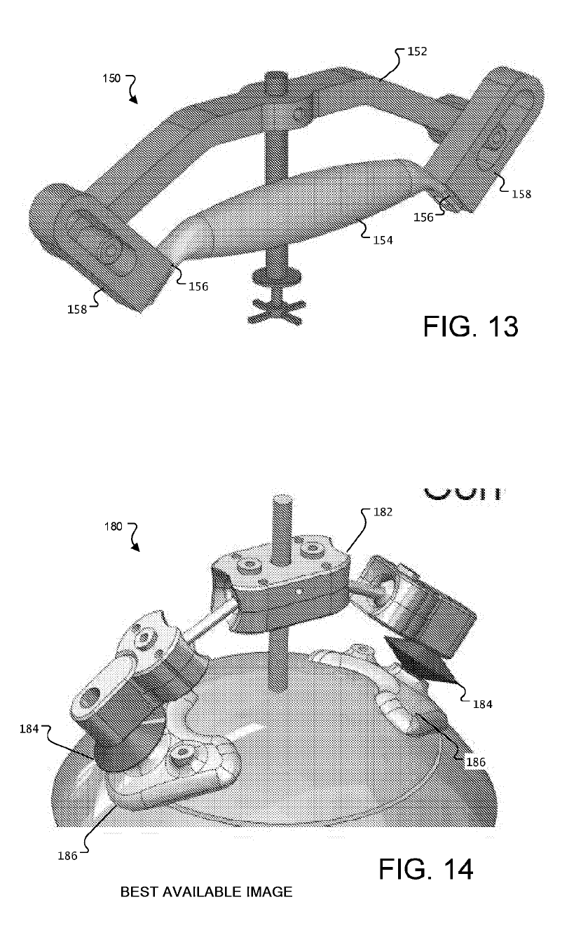

Referring to FIG. 13, in an alternative ventricular assist device 150, a brace 152 houses a single, inflatable actuator 154. For instance, the actuator 154 can be a pneumatic artificial muscle, such as a McKibben actuator, that contracts when actuated. Both ends 156 of the actuator 154 are fixed to rotating components 158 on the brace 152 such that the actuator 154 is slightly curved. When the actuator 154 contracts upon actuation, the ends 156 of the actuator 154 move closer together, causing the rotating components 158 to move downwards and inwards. When inserted in the heart, this motion of the rotating components 158 results in a compressive force being exerted on the right ventricle free wall.

Referring to FIG. 14, in an alternative ventricular assist device 180, a brace 182 houses multiple extendible actuators 184 that extend in one dimension upon actuation. The actuators 184 are attached to plates 186 that contact the right ventricle free wall. When the actuators 184 extend upon actuation, the plates 186 are pressed into the right ventricle free wall, thus applying a compressive force to the right ventricle free wall.

In some examples, a ventricular assist device can include two braces connected to opposite ends of a shaft. The first end of the shaft penetrates the right ventricle free wall and is connected to a brace having actuators that can exert a compressive force on the right ventricle free wall. The second end of the shaft penetrates the left ventricle free wall and is connected to a brace having actuators that can exert a compressive force on the left ventricle free wall. In this configuration, each ventricle free wall can be compressed independently from the other free wall, thus enabling the ventricular assist device to be used to exclusively augment one ventricle or the other, or both, depending on the needs of the patient.

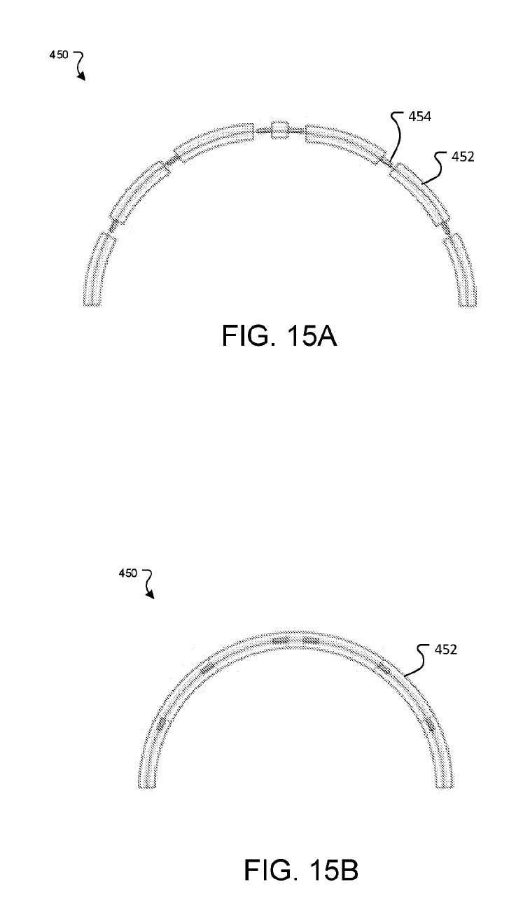

Referring to FIGS. 15A and 15B, a foldable ventricular assist device 450 is formed from multiple sub-pieces 452 that fit together to form the arc shape of the self-assembled ventricular assist device 450. A channel is formed through each sub-piece 452 and a narrow, flexible member 454 is threaded through the channel of each sub-piece. The narrow, flexible member 454 can be a metal wire or a string, e.g., of nitinol or stainless steel, e.g., 0.006 gauge. When tension is applied to the narrow, flexible member 454, the sub-pieces 452 are pulled together into the arc shape of the foldable ventricular assist device 450.

The self-assembly mechanism of the foldable ventricular assist device 450 enables the foldable ventricular assist device 450 to be inserted through a smaller incision, as each sub-piece 452 can be discretely inserted into the body with a large amount of flexibility between each pair of sub-pieces 452. For instance, without actuators, the foldable ventricular assist device 450 can be deployed through a 4 cm incision with approximately 3.5 cm of rib spreading. With two long actuators attached thereto, the foldable ventricular assist device 450 can be deployed through a 5 cm incision with approximately 4 cm of rib spreading.

### EXAMPLES

The invention is further described in the following examples, which do not limit the scope of the invention described in the claims.

The following examples generally show fabrication and results of in vivo testing of ventricular assist devices, demonstrating the ability of these devices to augment blood flow from ventricles.

### Example—Fabrication of Ventricular Assist Devices

An example ventricular assist device was fabricated using an actuator based on a McKibben pneumatic artificial muscle. A thermoplastic elastomer (TPE) bladder (Stretchlon 200, Airtech International, USA) was fabricated using a heat press and former. A polyurethane airline was bonded in to the base of the TPE bladder and the entire assembly was encapsulated within a mesh of 1″ dimeter. A rubber outer on the actuator was used to enable rapid recoil back during diastole to allow refilling of the heart.

The semilunar frame for the brace of a right ventricular assist device was a turned aluminum disc to which the soft actuators were affixed. For a left ventricular assist device, three-dimensional polyjet printing (Connex, Stratsys) was used to fabricate the brace. The brace bar was fabricated from polyether-ether-ketone (PEEK) to reduce the volume of metal, thus reducing noise artifacts from in vivo ultrasound imaging. The septal anchor and disc assemblies were produced from PEEK and stainless steel to tolerate the dynamic mechanical loading during device operation.

A custom electro-pneumatic control system was developed to actuate the ventricular assist devices. For right ventricular assist devices, a pacemaker was used to simultaneously pace the heart and provide an input to the control system. For left ventricular assist devices, a pressure sensing catheter in the left ventricle was used to detect the end of diastole and the beginning of systole. The signal (pacemaker or ventricular pressure) was acquired through analog input module (NI 9205, National Instruments, USA) and processed by a real-time controller (cRIO 9030, National Instruments), which generated output signals to trigger up to three pneumatic valves (NVKF333-5G-01T, SMC Corporation, USA).

The control system permitted the inflow valves to be opened for an arbitrary time period and after an arbitrary time delay following the input pacemaker or ventricular pressure signal. The control software can be configured to modulate the actuation duration period as a percentage of the cardiac cycle according to the instantaneous heart rate. A host computer was used to communicate the timing variable values to the real-time control system.

The control system received an air pressure and a vacuum supply and the valves were configured to switch to provide either vacuum or pressure to the actuators. A regulator (ITV series, SMC Corporation) was used to regulate the air pressure and was controlled by the real-time control system. The host computer provided a graphical user interface of the instantaneous regulator pressure, trigger signal, and valve timing configuration.

### Example—In Vivo Right Ventricle Testing of Ventricular Assist Devices

The functionality of ventricular assist devices was tested in an in vivo study in am 80 kg Yorkshire swine. A midline sternotomy was performed to access the heart of the swine. Flow probes (16PS and 20PS, Transonics Corporation, USA) were placed over the pulmonary artery and the aorta to measure blood flow. Pressure transducers (Surgivet Inc, Smiths Medical, USA) were used for direct measurement of blood pressure in the right ventricle, left ventricle, and the pulmonary artery. The pressure transducer signals, ECG and end tidal CO2 signals were passed to a clinical monitoring system (Surgivet, Smiths Medical, USA).

Initial baseline readings were obtained for a healthy heart and following heart failure. Ejection volumes for each cycle were computed by integrating the pulmonary artery and aortic flow rate data using analysis software (LabChart, AD Instruments, New Zealand). Individual pulmonary artery and aortic flow rates were computed for each cardiac cycle by multiplying the ejection volume by the instantaneous heart rate.

A ventricular assist device was placed in the heart of the swine using three-dimensional echocardiography guidance. Standard dosing of heparin, which is used during intracardiac device deployment procedures (150-300 U/kg, ACT time above 250 sec), was used during the deployment of the septal anchoring system and brace bar to minimize the risk of a thromboembolic event occurring. The cardiac output resulting from the device over an extended period of operation was characterized. Consecutive cardiac cycles in heart failure were considered immediately after activation of the device and again after five minutes of operation. For the right ventricle study, ten consecutive cycles were used. For the left ventricle study, fifteen consecutive cycles were used. Individual volume ejections from the pulmonary artery and aorta, RV and LV pressures (systolic and diastolic), aortic pressure (systolic and diastolic), pulmonary artery pressure (systolic and diastolic) and end tidal CO2 were logged. Normality tests were performed on the data set using histograms. A one-way analysis of variance (ANOVA) was performed to determine statistical significance with Tukey's post hoc test, considering p<0.05 to be statistically significant.

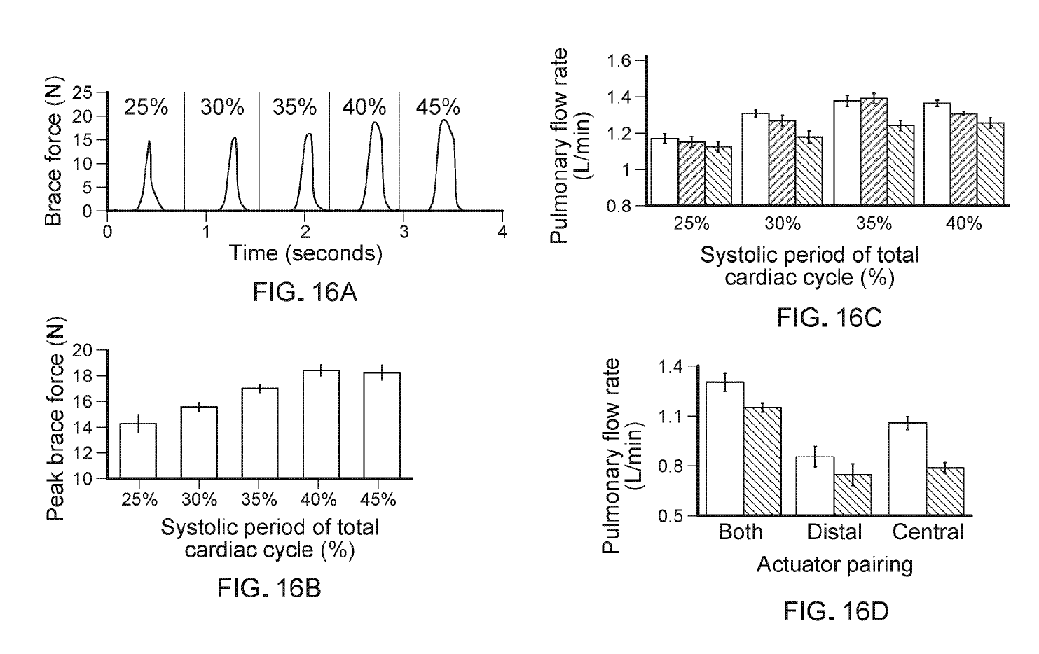

In an in vivo experiment, cardiac assistance to the right ventricle using a ventricular assist device with two inflatable actuators was assessed through an in vivo porcine test. Ventricular pacing was used to disrupt the native heart rhythm, causing acute heart failure. The pacemaker signal was also used as a control input to trigger contraction of the soft actuators in synchrony with the heart. Referring to FIGS. 16A-16E, measurements of blood pressure and flow were made at a baseline condition, in heart failure, and during device actuation at various operating conditions. FIG. 16A is a plot of force profiles for different systolic timings as a percentage of the total cardiac cycle. FIG. 16B is a plot of peak reaction forces observed for different systolic timings. FIG. 16C is a plot characterizing the pulmonary flow output for the actuation of the ventricular assist device at different systolic timing periods and delay periods. FIG. 16D is a plot showing the contribution of the actuator pairings on pulmonary flow rate. The error bars denote +/− standard deviation.

The force exerted on the right ventricle by the ventricular assist device was assessed using a modified semilunar bracing frame with integrated force sensors to quantify the total axial force transmitted to the septum during operation. Peak force measurements of 14-18 N were observed (FIG. 16B).

The effect of altering actuation timing conditions on pulmonary flow output was studied to achieve target synchronization between the heart and the ventricular assist device. The systolic actuation period (defined as a fraction of the total cardiac cycle) and the time delay after the initial pacemaker input were varied. During a state of right heart failure, the device was actuated for all combinations of systolic periods (25, 30, 35 and 40% of the cardiac cycle period) and delay periods of 0, 5 and 10% of the total cardiac cycle period (FIG. 16C). The systolic actuation period was shown to be a more significant factor for maximizing pulmonary flow than delay period, with 35% being the optimum period for a 96 bpm heart rate. The results indicate that the device is tolerant to timing delay discrepancies of up to 5% of the cardiac cycle at 96 bpm and 35% systolic actuation period. Otherwise, the addition of delays in to the control system resulted in a loss of pulmonary flow output.

The contribution of the different actuator pairings and use of the recoiling bands was studied. The results demonstrate that the use of all four actuators provide good performance on the right ventricle (FIG. 16D). The central actuators provided the greatest contribution when bands were present, although the combination of both pairs of actuators was key under both conditions. Without being bound by theory, it is believed that by inflating all actuators simultaneously, a larger proportion of the ventricle is compressed which minimizes local ballooning of the RV and forces blood to be ejected through the pulmonary artery. Overall, when the elasticated bands were attached to recoil the sealing ring, pulmonary flow rate was significantly augmented.

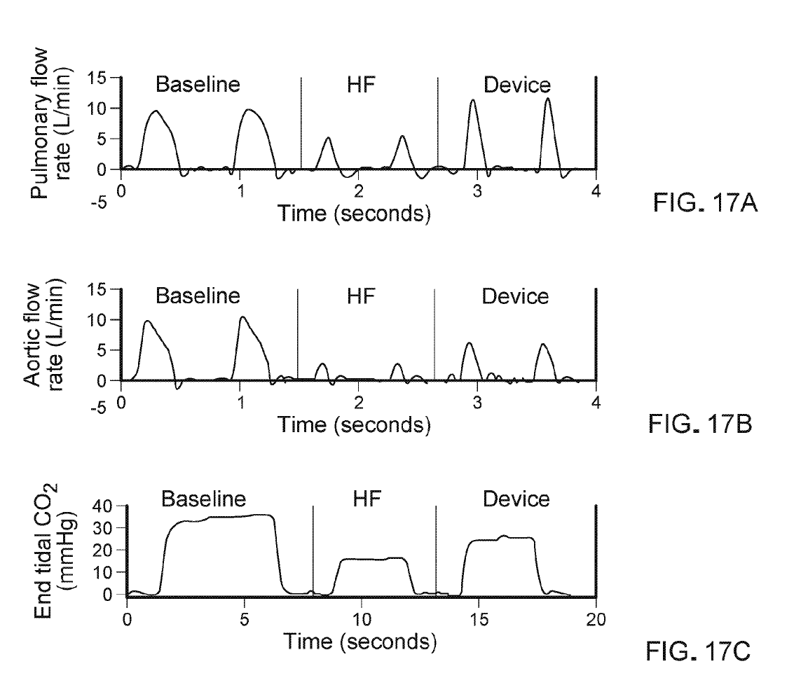

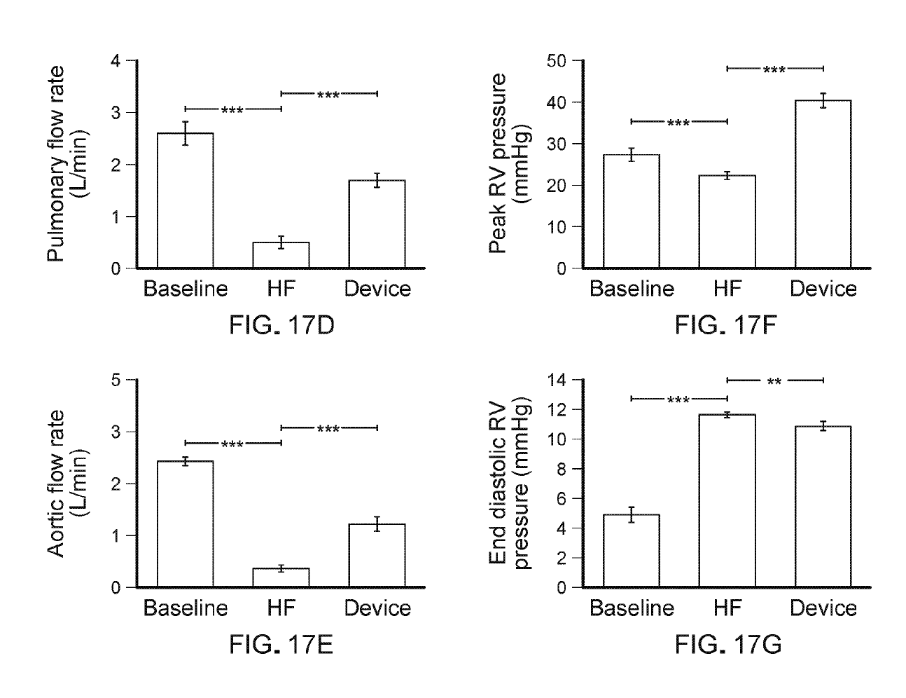

Referring to FIGS. 17A-17G, the ventricular assist device was assessed after 5 minutes of operation to account for transient effects after initial actuation in simulated right heart failure. FIGS. 17A-17C are plots showing the pulmonary flow rate, aortic flow rate, and end tidal CO2, respectively, versus time at baseline, heart failure, and with the device actuated. FIGS. 17D-17G are plots showing the pulmonary flow rate, aortic flow rate, peak right ventricle pressure, and end diastolic right ventricle pressure at baseline, heart failure, and with the device actuated, for ten consecutive cycles. Error bars denote +/− standard deviation. *** denotes p<0.001. ** denotes p<0.1.

The pulmonary flow rate was reduced to 19.3% of the baseline flow rate (2.59 L/min) in the right heart failure condition. After actuation of the device, a pulmonary flow rate of 1.7 L/min was observed which corresponds to a 66% recovery of the baseline level (FIG. 17A, 17D). The improved effect was also seen on the left side of the heart with a net improvement of 0.85 L/min in aortic flow from the heart failure baseline (FIG. 17B, 17E). End tidal CO₂ represents the maximal concentration of carbon dioxide at the end of exhalation and is directly related to pulmonary blood flow. End tidal CO₂ was markedly improved following actuation of the device (FIG. 17C). Induction of heart failure caused a drop in systolic right ventricle pressure and increased the end diastolic right ventricle pressure (FIG. 17D, 17E). After device actuation, systolic right ventricle pressure was augmented (p<0.001) whilst end diastolic right ventricle pressure was reduced (p<0.001).

### Example—In Vivo Right Ventricle Testing of Ventricular Assist Devices

The ability of the ventricular assist device to augment cardiac function was tested in the left ventricle in an in vivo porcine study. To simulate the left heart failure, coronary arteries ligation procedure was performed to create ischemia in a separate porcine model. After ligation, the aortic flow rate was reduced from 2.62 L/min to 1.03 L/min (p<0.001). The soft robotic device was actuated and cardiac output was assessed after 5 minutes of operation to negate transient effects. A pressure sensing catheter introduced into the LV was used to trigger the device to actuate at the end of diastole. The device was actuated at a systolic actuation period of 40% of the cardiac cycle, with zero delay. The heart rate was 109.7 bpm during device operation (up from 81.5 bpm) in the healthy baseline condition.

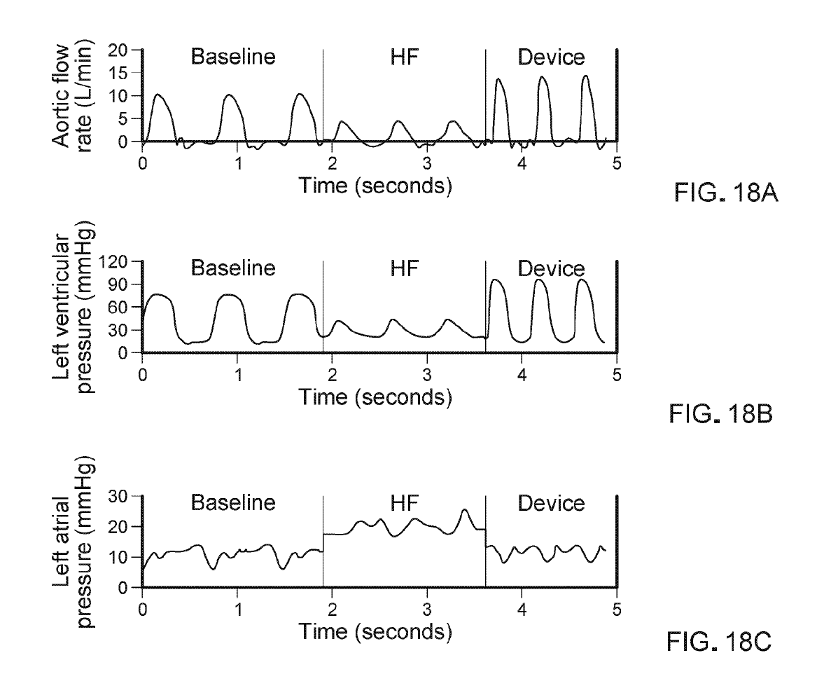

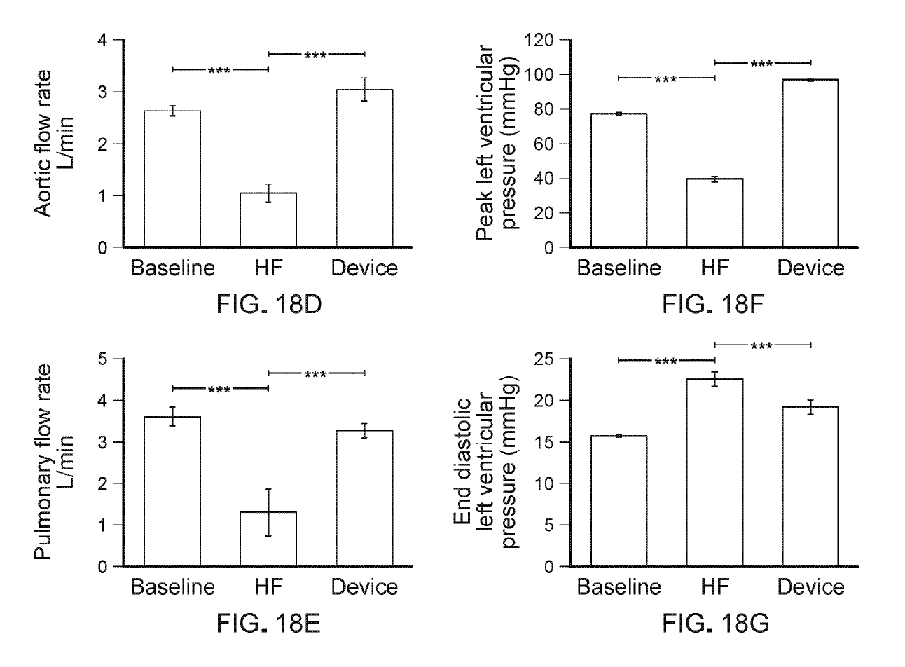

FIGS. 18A-18C are plots showing the aortic flow rate, left ventricle pressure, and left atrial pressure versus time at baseline, heart failure, and with the device actuated. FIGS. 18D-18G are plots showing aortic flow rate, pulmonary flow rate, peak left ventricle pressure, and end diastolic left ventricle pressure at baseline, heart failure, and with the device actuated, for 15 consecutive cycles. Error bars denote +/− standard deviation. *** denotes p<0.001.

Aortic flow was significantly augmented from the heart failure condition (p<0.001) and restored to 116% of the healthy baseline (FIG. 18A, 18D). Similarly, peak left ventricle pressure and mean pulmonary flow were augmented from the left heart failure condition to levels close to the healthy baseline (FIG. 18B, 18E, 18F). Both the mean left atrial pressure and end diastolic left ventricle pressure dropped during actuation (FIG. 18C, 18G), which implies improved diastolic function of the left ventricle.

### Example—Foldable Ventricular Assist Devices

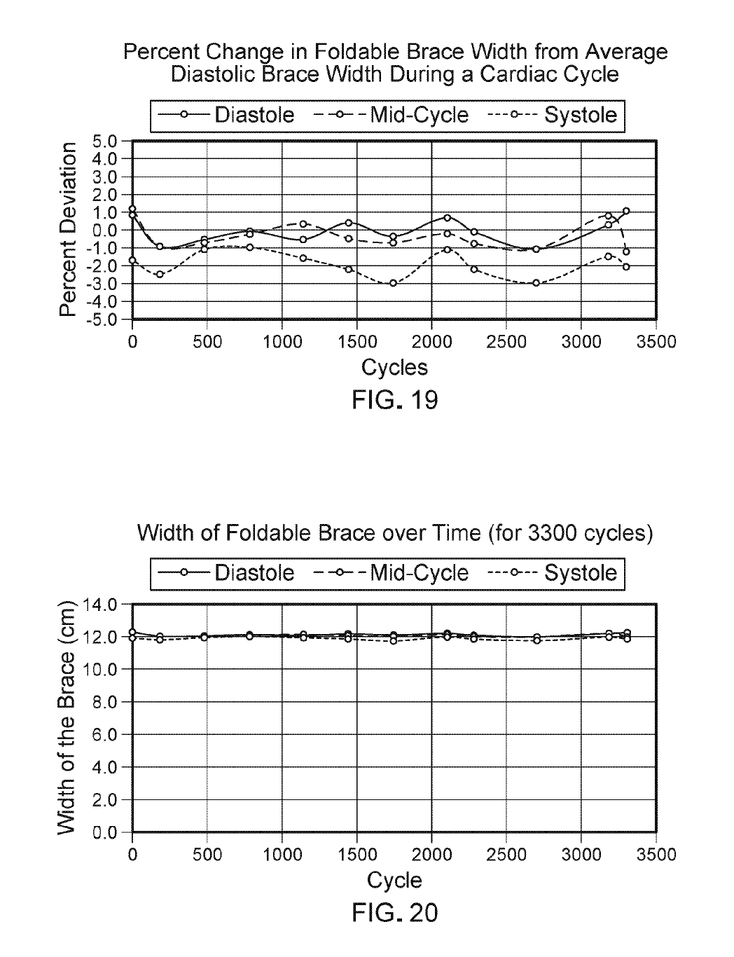

Simulations were performed using a surgical simulator model of the thoracic cavity. The simulation included simulated device insertion through an incision between two of the lower ribs on the patient's right side. Referring to FIG. 19, the simulated performance of the foldable ventricular assist device was compared to the simulated performance of a ventricular assist device with a solid, one-piece brace (referred to as a one-piece ventricular assist device). The plot of FIG. 19 shows the percent change in the width of the foldable ventricular assist device from the average width of a one-piece device during a cardiac cycle.

In vitro performance testing was performed to test the performance of a foldable ventricular assist device. A three-dimensional (3-D) printed replica of the brace was used for this experiment. The phantom heart for this experiment was a standard saline IV bag filled with water, connected to a tube with graduated markings, allowing for viewing of the relative volume ejection of fluid from the bag.

Referring to FIG. 20, the performance of a foldable ventricular assist device was monitored over 1 hour (3600 cycles, 60 beats per minute, with 12 psi in the actuators) in an in vitro situation. No plastic deformation of the brace was observed. Referring to FIG. 21, the performance of foldable ventricular assist devices was generally comparable to the performance of one-piece ventricular assist devices over time. At psi 13 in the actuators, the actuators burst before the end of the simulation. At psi 12, comparable performance was observed between the two devices. Although over time the simulated ejection fraction decreases, it is believed that this decrease may be due to experimental conditions.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A ventricular assist device comprising:
a rigid, elongated shaft;
an anchor assembly attached to a first end of the elongated shaft;
a brace attached to a second end of the elongated shaft, the brace having a surface facing the anchor assembly; and
one or more actuators attached to the brace and disposed adjacent the first surface of the brace.

2. The ventricular assist device of claim 1, wherein the one or more actuators are configured to expand towards the anchor assembly when actuated.

3. The ventricular assist device of claim 1 or 2, wherein the brace has an arc shape.

4. The ventricular assist device of any of the preceding claims, wherein the anchor assembly comprises an anchor having multiple, collapsible arms.

5. The ventricular assist device of claim 4, wherein the collapsible arms have a first configuration in which the collapsible arms are collapsed along a central post of the anchor and a second configuration in which the collapsible arms are disposed away from the central post of the anchor.

6. The ventricular assist device of claim 4 or 5, wherein the anchor comprises a central post.

7. The ventricular assist device of claim 6, wherein the anchor assembly comprises a disc attached to the central post of the anchor.

8. The ventricular assist device of any of the preceding claims, wherein the one or more actuators comprise inflatable actuators.

9. The ventricular assist device of any of the preceding claims, wherein the one or more actuators comprise actuators configured to expand in one or more dimensions when actuated.

10. The ventricular assist device of any of the preceding claims, wherein the one or more actuators comprise actuators configured to bend in one or more dimensions when actuated.

11. The ventricular assist device of any of the preceding claims, wherein the one or more actuators comprise pneumatic artificial muscle.

12. The ventricular assist device of any of the preceding claims, further comprising a ring disposed along the shaft, wherein the shaft passes through a central opening of the ring.

13. The ventricular assist device of claim 12, further comprising a sealing component, wherein a first side of the sealing component is attached to the ring and a second side of the sealing component is attached to the shaft.

14. The ventricular assist device of claim 12 or 13, further comprising a recoil component connected to the brace and to the ring, wherein the recoil component is configured to apply a recoil force to the ring.

15. The ventricular assist device of claim 14, wherein the recoil force is in a direction opposite to a direction of the expansion of the one or more actuators.

16. The ventricular assist device of claim 14 or 15, wherein the recoil component comprises one or more of a spring and an elastic band.

17. The ventricular assist device of any of the preceding claims, further comprising a control system configured to control actuation of the one or more actuators.

18. The ventricular assist device of claim 17, wherein the control system is configured to control actuation of the one or more actuators based on a signal indicative of heart function.

19. The ventricular assist device of claim 18, wherein the signal indicative of heart function comprises one or more of a pacemaker signal, an electrocardiography signal, a pressure in a ventricle of the heart, a pressure in an atrium of the heart, and a pressure in a great vessel.

20. The ventricular assist device of any of the preceding claims, wherein the brace comprises multiple sub-pieces connected by a wire or string.

21. A ventricular assist device inserted in a heart of a patient, the ventricular assist device comprising:
an anchor assembly secured to a septum of the heart;
an elongated shaft, wherein a first end of the elongated shaft is attached to the anchor assembly, a length of the elongated shaft is disposed in a ventricle of the heart, and a second end of the elongated shaft is disposed outside of a free wall of the ventricle;
a brace attached to the second end of the elongated shaft; and
one or more actuators attached to the brace and disposed between the brace and the free wall of the ventricle.

22. The ventricular assist device of claim 21, wherein the one or more actuators are configured to apply a compressive force to the free wall of the ventricle when actuated.

23. The ventricular assist device of claim 22, wherein the compressive force applied to the free wall of the ventricle is sufficient to cause the free wall of the ventricle to move toward the septum.

24. The ventricular assist device of claim 22 or 23, wherein the compressive force applied to the free wall of the ventricle is sufficient to cause the ventricle to shorten along an axis of the ventricle.

25. The ventricular assist device of any of claims 21 to 24, wherein the ventricle is a first ventricle and wherein the anchor assembly comprises:
an anchor disposed along a side of the septum facing a second ventricle of the heart;
a disc disposed along a side of the septum facing the first ventricle of the heart; and
a central post connecting the anchor and the disc.

26. The ventricular assist device of any of claims 21 to 25, wherein the elongated shaft passes through an incision in the free wall of the ventricle.

27. The ventricular assist device of claim 26, further comprising a ring disposed in the incision, wherein the shaft passes through a central opening of the ring.

28. The ventricular assist device of claim 27, further comprising a recoil component connected to the brace and to the ring, wherein the recoil component is configured to apply a recoil force to the ring.

29. The ventricular assist device of any of claims 21 to 28, wherein the one or more actuators comprise inflatable actuators.

30. The ventricular assist device of any of claims 21 to 29, wherein the ventricle comprises the right ventricle.

31. The ventricular assist device of any of claims 21 to 29, wherein the ventricle comprises the left ventricle.

32. The ventricular assist device of any of claims 21 to 31, wherein the brace comprises multiple sub-pieces connected by a wire or string.

33. A method of using a ventricular assist device disposed in a heart of a patient, the method comprising:
actuating one or more actuators disposed outside of a free wall of a ventricle of the heart to apply a compressive force to the free wall of the ventricle, wherein the one or more actuators are attached to a brace that is coupled to a ventricular septum of the heart, wherein application of the compressive force causes one or more of (i) the free wall of the ventricle to move towards the septum and (ii) the ventricle to shorten along an axis of the ventricle; and
de-actuating the one or more actuators to remove the compressive force from the free wall of the ventricle.

34. The method of claim 33, wherein actuating the one or more actuators comprises inflating the one or more actuators.

35. The method of claim 33 or 34, wherein actuating the one or more actuators comprises expanding the one or more actuators in one or more dimensions.

36. The method of any of claims 33 to 35, wherein actuating the one or more actuators comprises bending the one or more actuators in one or more dimensions.

37. The method of any of claims 33 to 36, wherein the one or more actuators comprise pneumatic artificial muscle and wherein actuating the one or more actuators comprises contracting the pneumatic artificial muscle.

38. The method of any of claims 33 to 37, wherein the brace is rigidly coupled to the septum.

39. The method of any of claims 33 to 38, further comprising controlling the actuating and de-actuating of the one or more actuators based on a signal indicative of heart function.

40. The method of claim 39, comprising actuating the one or more actuators during diastole.

41. The method of claim 39 or 40, comprising de-actuating the one or more actuators during systole.

42. The method of any of claims 39 to 41, wherein the signal indicative of heart function comprises one or more of a pacemaker signal, an electrocardiography signal, and a pressure in a ventricle of the heart.

43. A method of inserting a ventricular assist device into a heart of a patient, the method comprising:
securing an anchor assembly to a septum of the heart;
attaching a first end of an elongated shaft to the anchor assembly, wherein a brace is attached to a second end of the elongated shaft and wherein the brace remains outside of a free wall of the ventricle, and wherein one or more actuators attached to the brace are disposed between the brace and the free wall of the ventricle.

44. The method of claim 43, wherein inserting the elongated shaft into the ventricle of the heart comprises inserting the elongated shaft through a ring inserted into an incision in the free wall of the ventricle.
